(19) United States
(12) Patent Application Publication
(10) Pub. No.: US 2025/0302884 A1
(43) Pub. Date: Oct. 2, 2025

(54) SPECIFIC LIVER MICROTISSUE COMPRISING AT LEAST THREE DIFFERENT PHENOTYPES OF HEPATOCYTES, AND USES THEREOF

(71) Applicant: TREEFROG THERAPEUTICS, PESSAC (FR)

(72) Inventors: Maxime FEYEUX, TALENCE (FR); Dilyana Aleksandrova TODOROVA, TALENCE (FR); Elise Laure WARTER, TALENCE (FR)

(21) Appl. No.: 18/861,812

(22) PCT Filed: May 5, 2023
(86) PCT No.: PCT/EP2023/062011

§ 371 Date: Oct. 30, 2024

Related U.S. Application Data
(63) Continuation of application No. 18/072,161, filed on Nov. 30, 2022.
Application No. 18/072,161 is a continuation of application No. 18/072,191, filed on Nov. 30, 2022.

(30) Foreign Application Priority Data
May 6, 2022 (FR) FR2204314
May 6, 2022 (FR) FR2204315

(51) Int. Cl. A61K 35/407; A61K 9/50; A61P 1/16; C12N 5/071

(52) U.S. Cl. CPC A61K 35/407; A61K 9/5036; A61P 1/16; C12N 5/0671; C12N 2501/16; C12N 2501/998; C12N 2506/45; C12N 2509/00; C12N 2513/00; C12N 2533/74

(57) ABSTRACT

The invention relates to a specific liver microtissue comprising at least 3 different phenotypes of hepatocytes obtained from induced pluripotent stem cells encapsulated in a single three-dimensionally closed microcompartment. The invention also relates to a method for preparing such a liver microtissue and the uses thereof in the treatment or prevention of liver failure.

### TECHNICAL FIELD

The present invention relates to the treatment of liver failure by the use of liver microtissue obtained from specific cell microcompartments. The invention in particular relates to a particular liver microtissue comprising at least 3 different phenotypes of liver cells obtained from induced pluripotent stem cells encapsulated in a single three-dimensional closed microcompartment.

### PRIOR ART

The liver is one of the most complex organs in the human body. An integral part of the digestive system, the liver is constantly supplied with nutritious or toxic substances resulting from digestion. The processing of these substances by the liver is essential for the organism and has the following objectives:
storage and distribution of nutrients resulting from digestion
degradation of toxic substances
synthesis of most blood proteins, and
production of bile.

To perform these functions, the liver is composed of a wide variety of cells, such as hepatocytes, bile duct cells (cholangiocytes), stellate cells (Ito cells), Kupffer cells, mesenchymal stem cells and endothelial cells.

Hepatocytes are the most represented liver cells and are responsible for the majority of liver functions, from fatty acid synthesis to urea production to plasma protein synthesis.

Cholangiocytes are the polarized epithelial cells forming the walls of the bile ducts. They have a role in regulating the secretion of bile and in collecting it in order to transport it from the hepatocytes to the intestine.

The other cells provide good vascularization as well as signaling functions and interactions with other liver cells and with cells of the immune system.

When the liver can no longer perform its functions, it is called liver failure. The main causes of liver failure are viral infections, drug overdoses, immunological disorders, hereditary diseases or blood circulation disorders. When damage to liver function is irreversible, the recommended treatment is liver transplantation.

Thus, liver transplantation represents the standard treatment for people with end-stage liver disease.

Today, there are great difficulties in finding organ donors who can supply a liver of sufficient quality for a transplant.

Like any organ transplant, liver transplantation can only be performed if the liver is of good quality in order to avoid the risks associated with the transplant such as infections, cancers, prolonged immunosuppression and major surgery.

Today, only ⅔ of patients can benefit from a transplant and only when their quality of life has greatly deteriorated. In addition, the cost to be expected for a liver transplant is around €1 million (or $1 million) in the United States.

Faced with these problems, several innovative solutions have emerged.

Transplantation of isolated hepatocytes has appeared as an attractive approach, but clinical trials have remained few and inconclusive, limited by the poor survival, integration and expansion of isolated hepatocytes following graft in vivo, factors therefore limiting the short- and long-term therapeutic effects. In fact, hepatocytes isolated from donors have a very limited capacity for proliferation in vivo and in vitro. Moreover, the isolated hepatocytes put in culture tend to enter a process of dedifferentiation, thus decreasing the chances of obtaining a sufficient number of mature hepatocytes.

Although this technique makes it possible to treat a large number of patients, the administered dose is very often insufficient and presents a risk during the injection of the cells escaping into the general circulation.

This is mainly due to the difficulty for single cells to integrate within the liver, as well as poor quality induced by the preparation of the primary cells and their culture in vitro and the rejection of part of the hepatocytes despite immunosuppression.

Consequently, the low number of hepatocyte donors, the stability and the limited functionality of these hepatocytes constitute a barrier to their use

The development of protocols for the guided differentiation of pluripotent stem cells, that is to say, embryonic stem cells and induced pluripotent stem cells, has however made it possible to obtain an almost inexhaustible source of hepatocytes.

Although promising, hepatocytes derived from pluripotent cells are very difficult to cultivate on a large scale and generate disproportionate production costs. For example, the expected cost for the production of autologous liver grafts from induced pluripotent stem cells is around 9.7 million dollars.

To date, guided differentiation protocols do not make it possible to produce a variety of functional cell phenotypes, and in particular enough mature hepatocytes; the cells obtained retain the characteristics of fetal liver hepatocytes, in particular with a persistent expression of alpha-fetoprotein and low albumin production.

Nevertheless, certain protocols make it possible to obtain liver cells with better functional characteristics. These protocols remain complex and require steps of dissociation, reaggregation or co-cultures with in particular mesenchymal stem cells and endothelial cells. These additional steps add additional risks, increasing the total cost and decreasing the control of the finished product.

For an application in cell therapy, it is necessary to be able to adapt the existing methods in order to: i) obtain production with a limited number of steps in order to be effective on a large scale, ii) improve the integration of the grafted cells. The techniques developed today do not make it possible to produce liver microtissue obtained from induced pluripotent stem cells suitable for large-scale culture and presenting functional hepatocytes.

The current methods for producing liver microtissue are still too complex, have multiple steps and therefore a very high cost, and are difficult to scale up.

There is therefore a significant need for a solution allowing large-scale production of liver microtissue comprising several phenotypes of liver cells, which can be produced on a large scale and can be directly transplanted, to meet an essential demand for liver grafts.

The aim of the invention is therefore to meet all of these needs and to overcome the disadvantages and limits of the prior art.

### SUMMARY OF THE INVENTION

To meet this objective, the invention proposes a specific liver microtissue, suitable for uses in cell therapy and in particular in the fight against liver failure.

To this end, the invention relates to a three-dimensional liver microtissue comprising at least 3 different phenotypes of liver cells, said cells of the microtissue all having been obtained from induced pluripotent stem cells encapsulated in a single three-dimensional closed microcompartment.

Advantageously, the liver microtissue has sufficient cellular diversity to sustainably restore and/or improve liver function.

Preferably, the liver microtissue comprises at least immature hepatocytes, mature hepatocytes and cholangiocytes.

According to a preferred object of the invention, the liver microtissue comprises at least:
liver cells, of which at least 40% of the liver cells are cells expressing cytokeratin 19 (CK19), and
cells expressing CD73 and CD90.

Preferably, the cells expressing CD73 and CD90 are mesenchymal stem cells and the cells expressing CK19 are cholangiocytes.

Advantageously, the phenotypic composition of the liver microtissue is close to that of a healthy human liver.

Preferentially, the liver microtissue according to the invention comprises:
at least one lumen,
at least one cell of the microtissue in contact both with a lumen and with the medium outside the microtissue, and
at least one cell surrounded only by cells.

Advantageously, the organization of the liver microtissue is close to the organization of the liver tissue in development, which enables it in particular to promote its integration into the liver and the proper performance of the metabolic functions in the treated liver.

According to a particularly preferred embodiment, the liver microtissue is in an ovoid, cylindrical, spheroid or spherical or substantially ovoid, cylindrical, spheroid or spherical or ellipsoidal shape. Preferably, the liver microtissue is in an ellipsoidal shape.

Advantageously, the ellipsoidal shape of the microtissue makes it possible to promote the survival of the liver microtissue. Thus, a greater part of the liver microtissues is integrated by the treated liver.

Preferably, the liver microtissue according to the invention comprises at least one bile duct and/or at least one glycogen granule.

According to a preferred object of the invention, the liver microtissue comprises:
between 50 and 99% of liver cells, of which between 20 and 60% are cells expressing cytokeratin 19, and
between 1 and 20% of cells expressing CD73 and CD90.

According to one variant, the liver microtissue is a three-dimensional liver microtissue, the largest dimension of which is between 500 and 700 μm, and/or expressing CYP3A4 monooxygenase with an activity of at least 75,000 RLU per million cells and/or producing at least 18 μg of urea per million cells per 24 hours.

Advantageously, the activity of the CYP3A4 associated with urea production makes it possible to guarantee a liver microtissue comprising at least functional liver cells.

Preferably, the liver cells secrete at least 75 μg of albumin per million cells per 24 hours.

Advantageously, the liver microtissue exhibits metabolic activity similar to a healthy liver.

An object of the invention is also a set of several three-dimensional liver microtissues in a medium, of which at least one liver microtissue is a liver microtissue according to the invention. Preferably, at least 50% (by number) of the liver microtissues of the set of liver microtissues are liver microtissues according to the invention.

According to another aspect, the invention relates to a three-dimensional closed cell microcompartment comprising an outer hydrogel layer defining an inner part, said inner part comprising at least one liver microtissue according to the invention.

The microcompartment according to the invention makes it possible to guarantee a microenvironment suitable for the culture of pluripotent stem cells and for their differentiation into cells constituting the microtissue according to the invention. Indeed, such a microcompartment makes it possible to reproduce the in vivo conditions of the cellular microenvironment during liver organogenesis.

The invention also relates to a set of cell microcompartments according to the invention.

According to another aspect, the invention relates to a method for preparing a microcompartment or a set of microcompartments comprising at least the implementation of the following steps: producing a microcompartment comprising induced pluripotent stem cells, inducing cell differentiation within the microcompartment so as to obtain at least 3 different phenotypes of liver cells.

Finally, the invention relates to a liver microtissue according to the invention or a microcompartment containing it, or a set of liver microtissues according to the invention or a set of microcompartments containing them, for its use as a drug, preferably in the prevention or treatment of liver failure caused by diseases such as hepatic fibrosis and cirrhosis, steatosis, non-alcoholic steatosis, hepatitis, metabolic diseases of the liver, diseases related to secretion of factors VIII, alpha 1 antitrypsin, IX and/or VWF, Wilson's disease and hereditary hemochromatosis.

Other features and advantages will emerge from the detailed description of the invention and the following examples.

### BRIEF DESCRIPTION OF THE FIGURES

FIG. 1a is a comparative representation of the expression of the genes of interest (EOMES, CXCR4, HHEX, PROX1, AFP, ASGR1) during differentiation according to the differentiation protocol.

FIG. 1b is a comparative representation of the expression of the genes of interest (SOX17, FOXA2, TBX, HNF4A, ALB, KT18) during differentiation according to the differentiation protocol.

FIG. 1c is a comparative representation of the expression of the genes of interest (GATA4, HNF1B, SOX9, KT19, TAT) during differentiation according to the differentiation protocol.

FIG. 2 is a comparative representation of the expression of the genes of interest (EOMES, CXCR4, FOXA2, SOX17) according to the culture conditions.

FIG. 3 is a comparative representation of the expression of proteins of interest (SOX17, FOXA2) depending on the culture conditions, 5 days after the start of differentiation.

FIG. 4a is a comparative representation of the expression of the genes of interest (PROX1, TBX, AFP, KT18, KT19, HNF4A) according to the culture conditions.

FIG. 4b is a comparative representation of the expression of the genes of interest (HNF1B, SOX9, ASGR1, ALB, TAT)) according to the culture conditions.

FIG. 5 is a comparative representation of expansion factor for 30 days as a function of the culture conditions.

FIG. 6 is a graphic representation of albumin secretion during differentiation as a function of culture conditions.

FIG. 7 is a graphical representation of urea production during differentiation as a function of culture conditions.

FIG. 8 is a graphical representation of CYP3A4 activity as a function of culture conditions.

FIG. 9 is a set of images obtained by confocal microscopy of a microcompartment presenting hepatocytes.

FIG. 10a is a set of images obtained by confocal microscopy of a microcompartment from D-0 to D-9 after the start of differentiation.

FIG. 10b is a set of images obtained by confocal microscopy of a microcompartment from D-12 to D-30 after the start of differentiation.

FIG. 10c is a set of images obtained by confocal microscopy of a microcompartment on D-15 after the start of differentiation.

FIG. 10d is a set of images obtained by confocal microscopy of a microcompartment on D-20 after the start of differentiation.

FIG. 10e is a set of images obtained by confocal microscopy of a microcompartment on D-30 after the start of differentiation.

FIG. 11 is a set of images obtained by confocal microscopy of a microcompartment identifying certain liver cell phenotypes.

FIG. 12a is a set of images obtained by confocal microscopy:
of single-cell grafts on day 2 (recipients 1 and 2, top row) after transplantation into Balb/c mice,
of microtissue grafts on day 2 (recipients 4 and 5, bottom row) after transplantation into Balb/c mice.

FIG. 12b is a set of images obtained by confocal microscopy:
of single-cell grafts on day 6 (recipient 3, top row) after transplantation into Balb/c mice,
of microtissue grafts on day 6 (recipient 6, bottom row) after transplantation into Balb/c mice.

FIG. 12c is a set of images obtained by confocal microscopy of the tissue at the injection site with the vehicle alone (basal medium containing 50% matrigel) without a graft in Balb/c mice.

FIG. 12d is a set of images obtained by confocal microscopy of microtissues according to the invention prior to transplantation.

FIG. 13a is a graphical representation of A1AT (alpha-1 antitrypsin) secretion prior to transplantation of 2D differentiated single cells (2D) and microtissues according to the invention (Invention).

FIG. 13b is a graphical representation of A1T (alpha-1 antitrypsin) detection in Balb/c mouse serum pre-transplantation (day 0), on day 2 and on day 6 after transplantation with 2D differentiated single cells (SC), with microtissues according to the invention (MT) and with the vehicle alone (control).

### DETAILED DESCRIPTION OF THE INVENTION

### Definitions

For the purposes of the invention, “alginate” means linear polysaccharides formed from β-D-mannuronate and α-L-guluronate, salts and derivatives thereof.

For the purposes of the invention, “hydrogel capsule” or “hydrogel microcompartment” means a three-dimensional structure formed from a matrix of polymer chains, swollen using a liquid, preferentially water.

For the purposes of the invention, “human cells” means human cells or immunologically humanized non-human mammalian cells. Even when this is not specified, the cells, stem cells, progenitor cells and tissues according to the invention consist of or are obtained from human cells or from immunologically humanized non-human mammalian cells.

For the purposes of the invention, “embryonic stem cell” means a pluripotent stem cell of cells derived from the internal cell mass of the blastocyst. The pluripotency of the embryonic stem cells can be evaluated by the presence of markers such as the transcription factors OCT4, NANOG and SOX2 and surface markers such as SSEA3/4, Tra-1-60 and Tra-1-81. The embryonic stem cells used in the context of the invention are obtained without destroying the embryo from which they originate, for example using the technique described in Chang et al. (Cell Stem Cell, 2008, 2 (2)): 113-117). Alternatively, human embryonic stem cells may be excluded.

Within the meaning of the invention, “a cell surrounded only by cells” in a microtissue means a cell that is neither in contact with a lumen nor in contact with the outside of the microtissue.

For the purposes of the invention, the term “mutant cell” refers to a cell carrying at least one mutation.

For the purposes of the invention, “pluripotent stem cell” or “pluripotent cell” means a cell which has the capacity to form all the tissues present in the entire organism of origin, without however being able to form an entire organism per se. Human pluripotent stem cells can be called hPSC in the present application. These may in particular be induced pluripotent stem cells (IPSC or hiPSC for human induced pluripotent stem cells), embryonic stem cells or MUSE cells (for “multilineage-differentiating stress enduring”).

For the purposes of the invention, “induced pluripotent stem cell” means a pluripotent stem cell induced to become pluripotent by genetic reprogramming of differentiated somatic cells. These cells are in particular positive for pluripotency markers, such as staining with alkaline phosphatase and expression of the proteins NANOG, SOX2, OCT4 and SSEA3/4. Examples of methods for obtaining induced pluripotent stem cells are described in the articles by Yu et al. (Science 2007, 318 (5858): 1917-1920), Takahashi et al (Cell, 207, 131 (5): 861-872) and Nakagawa et al (Nat Biotechnol, 2008, 26 (1): 101-106).

Within the meaning of the invention, “progenitor cell” means a stem cell already engaged in the differentiation into liver cells but not yet differentiated.

According to the invention, “Feret diameter” of a microcompartment (or part of a microcompartment) or of a microtissue according to the invention means the distance “d” between two tangents to said microcompartment (or to said part) or of the microtissue, these two tangents being parallel, such that the entire projection of said microcompartment (or of said part) or of the microtissue lies between these two parallel tangents. A Feret diameter of the internal part of the microcompartment is measured between two interfaces of the internal part and the external layer of the microcompartment, that is, the distance “d” between two tangents to said internal part, these two tangents being parallel, such that the entire projection of said internal part is between these two parallel tangents.

Within the meaning of the invention, “variable thickness” of a layer means the fact that the layer for the same microcompartment or the same microtissue does not have the same thickness everywhere.

For the purposes of the invention, “microcompartment” or “capsule” means a partially or entirely closed three-dimensional structure containing several cells.

Within the meaning of the invention, “microtissue” or “liver microtissue” means a three-dimensional human tissue comprising at least liver cells and whose largest dimension is less than 1 mm.

Within the meaning of the invention, “medium” means an aqueous solution including cells or microtissues, compatible with the survival, development and/or metabolism of the cells. It may be a culture medium.

For the purposes of the invention, “convective culture medium” means a culture medium stirred by internal movements.

For the purposes of the invention, the term “mutation” means a genetic or epigenetic mutation, preferentially a functional mutation. It may in particular involve a point modification of the genetic sequence, a structural variant, an epigenetic modification, or a modification of the mitochondrial DNA.

The term “functional mutation” within the meaning of the invention refers to a transmissible genetic or epigenetic modification which confers a potential gain or loss of function or loss of potential function to a relevant mutant cell. It preferably involves a mutation causing a modification of the phenotype of the affected mutant cell. Very preferentially, it involves a change of the genomic and/or of the epigenomic sequence which alters the therapeutic potential of a population of cells, or by increasing the risk associated with the therapy produced, or by decreasing the benefit provided by the therapy produced.

According to the invention, “smallest dimension” of a microcompartment or of a cell layer means the value of the smallest Feret diameter of said microcompartment.

For the purposes of the invention, “lumen” means a volume of aqueous solution topologically surrounded by cells. Preferentially, its content is not in diffusive equilibrium with the volume of convective liquid present outside the microcompartment.

According to the invention, “smallest radius” of the internal part of a microcompartment means half of the value of the smallest Feret diameter of the internal part of the microcompartment.

According to the invention, “average radius” of the internal part of a microcompartment means the average of the radii of the smallest compartment, each radius corresponding to half the value of a Feret diameter of the internal part of the microcompartment.

According to the invention, “expansion rate at X days” means a measurement of cell proliferation at time t=X. The rate of expansion is measured by taking the ratio of the number of cells counted on day X of the culture divided by the number of cells at the start of the culture (day of encapsulation or day of placement in culture).

According to the invention, “large-scale culture” means a cell culture method suitable for a production batch of liver microtissue making it possible to treat at least 1 patient, preferably 10 patients, more preferably 100 patients, even more preferably more than 1,000 patients.

According to the invention, “functional phenotype” of a microtissue means the presence of mature hepatocytes characterized by the expression of albumin and the absence of expression of alpha-fetoprotein and cytokeratin 19.

According to the invention, “liver bud” means a cellular organization characterized by a cellular extension of the endoderm of the embryonic foregut that gives rise to the parenchyma of the liver and the bile duct. This is a particular conformation giving rise to hepatocytes and cholangiocytes.

### Liver Microtissue

The invention therefore relates to a three-dimensional liver microtissue comprising at least 3 different phenotypes of liver cells, all the cells of the microtissue all having been obtained from induced pluripotent stem cells encapsulated in a single three-dimensional closed microcompartment.

Preferably, the liver microtissue comprises at least 4 different phenotypes of liver cells, even more preferably at least 5.

Advantageously, the liver microtissue according to the invention has a significant cellular diversity making it possible to reproduce the liver microenvironment. The diversity and their proximity to the cells present in the liver microtissue allows a large number of cellular interactions thus cooperating in the performance of numerous metabolic and transport functions.

The cellular diversity found in the liver microtissue according to the invention is obtained from induced pluripotent stem cells in a single three-dimensional closed microcompartment. In this way, the different cell types can organize themselves within the microcompartment reproducing the liver microenvironment.

According to a preferred embodiment, all the cells of the microtissue have all been obtained by differentiation of induced pluripotent stem cells encapsulated in a single three-dimensional closed microcompartment, said induced pluripotent stem cells preferably all being of the same line.

Very preferably, all the cells of the microtissue have all been obtained from induced pluripotent stem cells encapsulated in a single three-dimensional closed microcompartment by a single differentiation method implemented in said microcompartment.

Preferentially, the induced pluripotent stem cells before differentiation in microtissue according to the invention form a cyst in the microcompartment. Thus, preferably, all the cells of the microtissue have all been obtained from at least one cyst of induced pluripotent stem cells encapsulated in a single three-dimensional closed microcompartment, preferably by differentiation, in particular by a single differentiation method implemented in said microcompartment.

According to a variant, the liver microtissue according to the invention can be obtained from stem cells, progenitor cells and/or cells capable of differentiating into liver cells.

Advantageously, the microenvironment of the microcompartment from which the microtissue according to the invention is obtained reproduces the conditions of liver organogenesis. Indeed, the microcompartment according to the invention makes it possible to limit the various physical and/or stress constraints and promotes the interactions between the various cell types within the microcompartment.

According to a particularly suitable embodiment, the liver microtissue according to the invention comprises at least immature hepatocytes, mature hepatocytes and cholangiocytes. Preferentially, it comprises at least:
immature hepatocytes, characterized by the expression of alfa-fetoprotein (AFP) and albumin (ALB) and the absence of expression of cytokeratin 19 (CK19)
mature hepatocytes characterized by the expression of albumin and the absence of expression of alfa-fetoprotein and cytokeratin 19
cholangiocytes characterized by the expression of cytokeratin 19 and the absence of expression of albumin and alfa-fetoprotein.

According to a variant, the liver microtissue according to the invention comprises hepatoblasts. Hepatoblasts can be characterized by the expression of alpha-fetoprotein, albumin and cytokeratin 19.

The presence of these liver cell phenotypes warrants a multifactorial effect on liver functions. This effect may restore or optimize certain liver functions.

In a particular embodiment, the liver microtissue comprises at least 50% mature and/or immature hepatocytes.

According to a particularly suitable embodiment, the liver microtissue comprises, within the liver cells, between 20 and 60% (by number) cells expressing cytokeratin 19.

Cells expressing cytokeratin 19 are preferentially cholangiocytes.

In the context of the invention, the liver cells are preferably chosen from mature hepatocytes, immature hepatocytes, hepatoblasts, cholangiocytes and mixtures thereof.

Liver microtissue may also comprise cells expressing CD73 and CD90. Cells expressing CD73 and CD90 are preferentially mesenchymal stem cells.

Mesenchymal stem cells are a cell population well known for its properties on tissue repair and regeneration, particularly of the liver. Advantageously, the presence of mesenchymal stem cells derived from induced pluripotent stem cells of the patient to be treated makes it possible to guarantee efficacy of the tissue repair of the liver exhibiting liver failure.

Thus in a particular embodiment, the microtissue according to the invention comprises at least:
hepatic cells, preferentially at least immature hepatocytes, mature hepatocytes, cholangiocytes, and
cells expressing CD73 and CD90, preferentially at least mesenchymal stem cells.

Preferably, the liver microtissue comprises at least:
liver cells, of which between 20% and 60% (by number) of the cells are cells expressing cytokeratin 19, and
cells expressing CD73 and CD90.

The liver microtissue preferentially comprises (percentages by number):
between 50 and 99% of liver cells, of which preferentially between 20 and 60% (by number) of the cells are cells expressing cytokeratin 19, and
between 1 and 20% of cells expressing CD73 and CD90.

The liver microtissue according to the invention can also comprise other cells, such as in particular Ito cells (stellate cells), Kupffer cells, endothelial cells, hepatoblasts. Thus, in another particular embodiment, the liver microtissue comprises:
mature hepatocytes, immature hepatocytes, hepatoblasts, cholangiocytes,
Ito cells and/or Kupffer cells and/or endothelial cells,
and possibly mesenchymal stem cells.

According to a variant, the liver microtissue according to the invention can also comprise smooth muscle cells and/or fibroblasts.

The cell phenotypes comprised in the liver microtissue are preferably compatible with the liver microenvironment.

The cellular diversity offered by the liver microtissue according to the invention makes it possible to repair and regenerate the diseased liver so as to restore the affected functions.

The concentration of mature hepatocytes, cholangiocytes and mesenchymal stem cells ensures a lasting effect during cell therapy. It is particularly important when culturing to obtain a content of mature and/or immature hepatocytes greater than 50% in order to obtain a sufficient effect as soon as possible after microtissue graft.

A concentration of mature and/or immature hepatocytes of less than 50% would limit the efficiency of the graft and would require a graft volume that is all the greater as the concentration (by number) of hepatocytes is low. Indeed, it is estimated that at least 5% of the mass of the liver in functional hepatocytes is necessary to treat acute liver failure, for example, and metabolic deficiencies of the liver such as diseases linked to the secretion of factor VIII, factor IX and VWF, Wilson's disease and hereditary hemochromatosis require the same order of magnitude. Mature hepatocytes express albumin but do not express alpha-fetoprotein. These markers are easily identifiable and quantifiable by detection methods well known to those skilled in the art, such as flow cytometry.

Particularly preferably, the liver microtissue comprises at least one lumen, at least one cell of the microtissue in contact both with a lumen and with the medium outside the microtissue, and at least one cell surrounded only by cells.

In the context of the invention, such an organization is characteristic of a functional microtissue ready to be used in cell therapy.

The particular organization of the microtissue is possible in particular owing to the presence of polarized liver cells making it possible to structure and organize the microtissue.

Thus, the microtissue may contain polarized liver cells. Polarization of liver cells in the microcompartment may be an indicator of functional microtissue formation.

The liver microtissue according to the invention does not comprise human embryonic stem cells.

The liver microtissue according to the invention may be in the form of an ellipsoid.

Advantageously, the ellipsoidal shape of the microtissue makes it possible to promote the survival and integration of the liver microtissue. When the liver microtissue is injected into the general circulation, its ellipsoidal shape allows it to facilitate its flow into the blood vessels in the case of administration by injection via the vascular route (in an embodiment via the portal vein), but also to facilitate its flow within a cannula if the administration is done by intra-tissue graft. The improvement in the injection of the liver microtissue causes an improvement in the integration of the liver microtissue by the liver of the treated patient.

The liver microtissue according to the invention preferably has a diameter or a smaller dimension of between 100 and 300 μm, preferably between 150 and 280 μm. The largest dimension of the liver microtissue is preferably less than 1 mm, and is very preferably between 500 and 700 μm.

Advantageously, the size of the liver microtissue is adapted for administration by the portal vein.

The liver microtissue according to the invention may comprise between 300 and 14,000 cells, preferably between 500 and 8,000, even more preferably between 900 and 5,000, in particular 4,500 cells.

Preferably, the liver microtissue comprises at least one bile duct. The bile ducts collect the bile produced by the liver cells to transport it to the gallbladder. The presence of at least one bile duct within the liver microtissue promotes the proper functioning of the microtissue within the liver and the reconstruction of defective bile ducts.

According to another embodiment, the liver microtissue comprises at least one glycogen granule. Glycogen serves as storage for carbohydrates in the body; it is mainly stored in the liver. Under the action of insulin, liver cells store glucose in the form of glycogen. Under the action of glucagon, liver cells will hydrolyze glycogen and release glucose into the blood. The presence of glycogen granules in the liver cell is a physiological element of the functioning of its metabolism and therefore of its function. Thus, the presence of at least one glycogen granule in the liver microtissue guarantees favorable conditions for the functioning of the liver microtissues for an optimal therapeutic effect. Preferably, the liver microtissue comprises at least one bile duct and at least one glycogen granule.

According to one embodiment, the liver microtissue according to the invention is a three-dimensional liver microtissue, the largest dimension of which is between 500 and 700 μm. Preferentially, the liver microtissue according to the invention expresses CYP3A4 monooxygenase with an activity of at least 75,000 RLU per million cells and/or produces at least 18 μg of urea per million cells per 24 hours.

Cytochromes P450 are hemoproteins participating in the oxidative metabolism of many molecules. Cytochromes P450 are enzymes involved in the biotransformation of exogenous compounds, both in the phenomena of detoxification and intoxication by formation of reactive entities. The most abundant human hepatic form (CYP3A4) is responsible for the metabolism of more than 60% of drugs. Its presence within the microcompartment is a functional guarantee. Urea is a nitrogenous product resulting from the catabolism of proteins. It is exclusively synthesized in the liver via the urea cycle and the amount of urea formed depends on the amount of protein ingested, protein catabolism and the state of liver function. A CYP3A4 activity of at least 75,000 RLU per million cells associated with a urea production of at least 18 μg per million cells per 24 hours guarantees the presence of mature hepatocytes as well as sufficient metabolic activity to guarantee a significant effect on impaired liver functions.

Preferably, the activity of CYP3A4 is at least 80,000 RLU, even more preferably at least 100,000 RLU.

According to a preferred embodiment, the liver microtissue produces at least 40 μg of urea per million cells per 24 hours, even more preferably at least 60 μg, in particular at least 80 μg

Advantageously, the liver microtissue according to the invention exhibits a metabolic activity close to that of a healthy liver. Thus, liver microtissue is particularly effective in restoring the functions of the treated liver.

Preferably, the liver microtissue according to the invention comprises between 50 and 99% liver cells. The liver microtissue preferentially comprises liver cells secreting at least 75 μg of albumin per million cells per 24 hours, even more preferentially at least 150 μg, in particular at least 250 μg.

According to a particular embodiment, the liver microtissue is obtained from induced pluripotent stem cells and comprises liver cells secreting at least 75 μg of albumin per million cells per 24 hours at least 20 days after the start of differentiation.

Albumin is the most abundant protein in the blood. Produced by the liver, albumin is in particular responsible for stabilizing blood pressure and transporting many substances.

Advantageously, an albumin production of at least 75 μg per million cells per 24 hours is an indicator of proper functioning of the liver microtissue.

The liver microtissue according to the invention can be obtained by differentiation of induced pluripotent stem cells at least 20 days, preferably at least 30 days after encapsulation in a three-dimensional closed microcompartment of 1 to 200 induced pluripotent stem cells, preferably between 5 and 150, especially between 15 and 80.

Preferably, the liver microtissue can be obtained from the encapsulation in a three-dimensional closed microcompartment of 1 to 200 induced pluripotent stem cells, preferably between 5 and 150, in particular between 15 and 80.

Preferably, the induced pluripotent strains form a cyst in the microcompartment before differentiation into cells of the liver microtissue according to the invention.

An object of the invention is also a set of liver microtissues comprising at least one liver microtissue according to the invention. Preferably, it is a set of several three-dimensional liver microtissues in a medium, of which at least one liver microtissue is a liver microtissue according to the invention. According to a preferred variant, at least 50% (by number) of the liver microtissues of the set of liver microtissues are liver microtissues according to the invention.

The set of liver microtissues according to the invention is adapted to be administered to patients with liver failure. Administration can be by injection in a biocompatible solution. The injection can be carried out in the portal vein so as to reach the liver without dispersion in the general circulation, directly in the liver or ectopically. When the injection is ectopic, it can be carried out in the abdomen or under the renal capsule.

Preferably, the effective quantity of the set of microtissues injected into the patient corresponds to a mass between 1 and 20% of the mass of the liver of the treated patient, preferably between 2 and 10%, in particular 5%.

According to another aspect, the object of the invention is a microcompartment comprising at least one liver microtissue according to the invention.

The microcompartment according to the invention comprises an external hydrogel layer. Preferentially, the hydrogel used is biocompatible, that is to say it is non-toxic to the cells. The external hydrogel layer must allow the diffusion of oxygen and nutrients in order to supply the cells contained in the microcompartment and to enable them to survive. According to one embodiment, the external hydrogel layer comprises at least alginate. It may consist exclusively of alginate. The alginate can in particular be a sodium alginate, composed of 80% α-L-guluronate and 20% β-D-mannuronate, with an average molecular weight of 100 to 400 kDa and a total concentration of between 0.5 and 5% by weight. The external hydrogel layer has no cells.

The outer hydrogel layer makes it possible in particular to protect the cells from the mechanical stress of bioreactors, to limit potentially toxic molecules when accumulated in the medium.

The average outer layer thickness can be variable. It is preferably between 20 and 60 μm, more preferably between 30 and 40 μm. The ratio between the smallest radius of the inner part and this thickness is preferably between 2 and 10 μm.

The microcompartment according to the invention advantageously exhibits a rate of expansion of the induced pluripotent stem cells of at least 15 times, 20 days after the start of differentiation.

The invention thus promotes amplification with a high rate of expansion, which consequently reduces the culture time to obtain a functional microtissue.

The microcompartment according to the invention can be obtained after encapsulation of induced pluripotent stem cells with or without addition of extracellular, natural or synthetic matrix.

According to a variant, the microcompartment according to the invention comprises, in its inner part, extracellular matrix such as Matrigel® and/or Geltrex® and/or a hydrogel-type matrix of plant origin such as modified alginates or of synthetic origin or a copolymer of poly(N-isopropylacrylamide) and poly(ethylene glycol) (PNIPAAm-PEG) of the Mebiol® type.

According to a variant, the microcompartment according to the invention can be obtained after encapsulation of induced pluripotent stem cells without addition of extracellular matrix. The extracellular matrix elements can be peptide or peptidomimetic sequences, mixtures of proteins, extracellular compounds or structural proteins, such as collagen, laminins, entactin, vitronectin, as well as growth factors or cytokines.

Within the microcompartment, the induced pluripotent stem cells differentiate into liver tissue for a period of at least 20 days, preferably at least 30 days.

Unexpectedly, the microcompartment provides a favorable microenvironment for the development of liver microtissue. Indeed, during the differentiation within the microcompartment, the cells organize themselves to form a structure similar to a liver bud. This liver bud is found during liver organogenesis in vivo; it is this particular structure that gives rise to hepatocytes and cholangiocytes. The presence of a structure similar to a liver bud during the formation of the liver microtissue is an indicator of the good quality of the tissue.

Thus, the liver microtissue according to the invention is preferably obtained after the formation of a structure similar to a liver bud in a single three-dimensional closed microcompartment.

The microcompartment according to the invention makes it possible to isolate the induced pluripotent stem cells from the mechanical stresses present within the bioreactor. This mechanical isolation allows the microtissue to set up and maintain a topology that approximates the spatial and structural organization of the liver organogenesis existing in vivo.

Unexpectedly, after at least 20 days, preferentially 30 days, of differentiation within the microcompartment, the cells retain their conformation, thus making it possible to have better proliferation while maintaining a functional phenotype. Consequently, this makes it possible to reduce the number of passages and to reduce the time in culture necessary to reach the final number of liver cells required.

The cells present in the microcompartment according to the invention were preferentially obtained after at least two cell division cycles after the encapsulation in an outer hydrogel layer of 1 to 200 induced pluripotent stem cells, preferably between 5 and 150, in particular between 15 and 80.

Preferably, the cells present in the microcompartment according to the invention have been obtained after at least 2, 3, 4, 5, 6, 7, 8, 9, 10, 11, 12, 13, 14, 15, 20, 25, 28, 30 cell division cycles after encapsulation in an outer hydrogel layer, preferably at least 5, even more preferably at least 6, to obtain up to 8,000 mature hepatocytes per microcompartment. For example, mature hepatocytes present in the microcompartment were obtained after at least six cell division cycles after encapsulation of cells in the outer hydrogel layer.

Preferentially, the number of cell divisions for the implementation of the method according to the invention is less than 100, even more preferentially less than 30.

Preferably, the microcompartment is obtained after at least 2 passes after encapsulation, more preferably at least 3, 4 or 5 passes. Each pass can for example last between 2 and 10 days, in particular between 2 and 4 days.

Preferably, the microcompartment is obtained after at least one re-encapsulation, more preferably between 1 and 14 re-encapsulations, in particular between 2 and 7 re-encapsulations. Very preferentially, a re-encapsulation corresponds to a new pass and each encapsulation cycle corresponds to a pass.

Preferably, the microcompartment according to the invention was obtained in less than 30 days after encapsulation, even more preferably in less than 20 days after encapsulation of at least 1 induced pluripotent stem cell in the inner part defined by the outer hydrogel layer, preferably 5, 20 and up to 100.

The microcompartment according to the invention can contain between 100 and 14,000 cells, preferably between 300 and 10,000 cells, even more preferably between 300 and 5,000, more particularly at least 50 mature hepatocytes and at least 20 cholangiocytes.

Advantageously, the microcompartment according to the invention protects the induced pluripotent stem cells from mechanical stress, thus making it possible to obtain a rate of expansion that is particularly suited to large-scale culture.

The microcompartment according to the invention can be in any three-dimensional form, that is, it may have the shape of any object in space. The microcompartment may have any form compatible with cell encapsulation. Preferably, the microcompartment according to the invention is in a spherical or elongated or ellipsoidal or substantially spherical or elongated shape. It may have the shape of an ovoid, a cylinder, a spheroid or a sphere or substantially this shape.

It is the outer layer of the microcompartment, that is the hydrogel layer, which imparts its size and shape to the microcompartment according to the invention. Preferably, the smallest dimension of the microcompartment according to the invention is between 150 μm and 500 μm, preferably between 200 μm and 450 μm.

Its largest dimension is preferably greater than 350 μm, more preferably between 350 μm and 600 μm.

The microcompartment according to the invention may optionally be frozen to be stored. It should then preferentially be thawed before it is used.

The invention also relates to several microcompartments according to the invention used together.

The invention also relates to an assembly or series of microcompartments comprising at least two three-dimensional cellular microcompartments, characterized in that at least one microcompartment is a microcompartment according to the invention.

Preferably, the series of microcompartments according to the invention is in a culture medium, in particular in an at least partially convective culture medium. Any culture medium suitable for culturing induced pluripotent stem cells can be used, such as for example the medium “HCM Hepatocyte Culture Medium BulletKit (Lonza)” or “Medium E from William (Thermofisher Scientific),” since the concentration of dissolved salts is compatible with the maintenance of alginate crosslinking by divalent cations.

According to a particularly suitable embodiment, the object of the invention is a series of cellular microcompartments as described above in a closed chamber, such as a bioreactor, preferentially in a culture medium in a closed chamber, such as a bioreactor. Thus, preferentially, the microcompartments are arranged in a culture medium in a closed bioreactor.

The set or series of microcompartments according to the invention preferably comprises between 2 and 10¹⁶ microcompartments.

Thus, the microcompartment according to the invention is suitable for large-scale culture by providing liver microtissues exhibiting a functional phenotype, in particular vis-à-vis their detoxifying (particularly the activity of CYP3A) and secretory (particularly the secretion of albumin) capacities, guaranteeing optimal efficacy in vivo.

### Use of Liver Microtissue According to the Invention

The liver microtissue or the microcompartment according to the invention can be used for all applications, in particular as a drug, in particular in cell therapy in humans.

Thus, the object of the invention is a liver microtissue according to the invention or a set of liver microtissues comprising at least one liver microtissue according to the invention, or a microcompartment according to the invention or a set of microcompartments comprising at least one microcompartment according to the invention, for its use as a drug, in particular in cell therapy.

The invention also covers a method of therapeutic treatment which consists of grafting and/or injecting microtissues according to the invention into humans, in particular as a cell therapy treatment.

Preferably, the liver microtissue or the microcompartment according to the invention can be used in the prevention or treatment of symptoms associated with liver failure, in particular acute, chronic or acute-on-chronic liver failure.

Preferably, the liver microtissue or the microcompartment according to the invention can be used in the prevention or treatment of disease such as fibrosis, cirrhosis, steatosis, non-alcoholic steatosis, hepatitis, metabolic diseases of the liver such as diseases linked to secretion of factor VIII and factor IX and VWF, Wilson's disease and hereditary hemochromatosis. Indeed, all these indications can be treated by liver transplantation, and this is precisely what the transplantation of a set of microtissues according to the invention synthesizing the liver functions is called upon to solve.

Thus, the object of the invention is a liver microtissue according to the invention or a set of liver microtissues comprising at least one liver microtissue according to the invention, or a microcompartment according to the invention or a set of microcompartments comprising at least one microcompartment according to the invention, for its use in the prevention or treatment of symptoms associated with liver failure, in particular acute, chronic or acute-on-chronic liver failure, in particular in the prevention or treatment of liver disease such as fibrosis, cirrhosis, steatosis, non-alcoholic steatosis, hepatitis, metabolic diseases of the liver such as diseases linked to secretion of factor VIII and factor IX and VWF.

The invention also relates to a liver microtissue according to the invention or a set of liver microtissues comprising at least one liver microtissue according to the invention, or a microcompartment according to the invention or a set of microcompartments comprising at least one microcompartment according to the invention, for its use in the evaluation of molecules or in the modeling of liver diseases.

### Method for Preparing Microcompartments and Microtissues According to the Invention

The invention also relates to a method for preparing microcompartments according to the invention.

The method for preparing a microcompartment or an assembly of microcompartments according to the invention may comprise the following steps:
(a) preparing a three-dimensional closed cell microcompartment comprising, inside an outer hydrogel layer, induced pluripotent stem cells, and optionally extracellular matrix elements or a natural or synthetic extracellular matrix,
(b) inducing cell differentiation within the cell microcompartment, so as to obtain at least 3 different phenotypes of liver cells.

The preparation of the microcompartment of step (a) can be carried out in any culture medium suitable for culturing induced pluripotent stem cells such as mTeSR™1 or mTeSRPlus media from Stemcell technologies, StemMACS™ IPS-Brew XF (Miltenyi Biotec or StemFlex from thermofisher Scientific).

The microcompartment of step (a) can be obtained by encapsulation of 1 to 150 induced pluripotent stem cells, preferably at least 50, in particular at least 100.

Preferably, the encapsulation is implemented according to techniques known to those skilled in the art. Indeed, any method of producing cell microcompartments containing inside an outer hydrogel layer and cells can be used to implement the preparation method according to the invention. In particular, it is possible to prepare microcompartments by adapting the method and the microfluidic device described in Alessandri et al., 2016 (“A 3D printed microfluidic device for production of functionalized hydrogel microcapsules for culture and differentiation of human Neuronal Stem Cells (hNSC),” Lab on a Chip, 2016, vol. 16, no. 9, pp. 1593-1604), in accordance with the steps described below.

Preferably, step (a) is implemented by adding extracellular matrix elements or a natural or synthetic extracellular matrix. According to a variant, step (a) is implemented without adding extracellular matrix elements or extracellular matrices or a natural or synthetic extracellular matrix.

In the context of the invention, step (a) is preferably implemented in a device capable of generating hydrogel capsules using a microfluidic chip. For example, the device may comprise syringe pumps for several solutions injected concentrically by virtue of a microfluidic injector, which makes it possible to form a jet which breaks up into droplets which are then collected in a calcium bath. According to a particularly suitable embodiment, two or three solutions loaded on two or three syringe pumps:
a hydrogel solution, for example alginate,
optionally an isotonic intermediate solution, preferentially an isotonic solution containing no divalent cations such as Ca2+Mg2+, to avoid excessively premature crosslinking of the hydrogel in the injector, such as for example a sorbitol solution,
a solution comprising induced pluripotent stem cells and culture medium.

The three solutions are co-injected (injected simultaneously) concentrically using a microfluidic injector or microfluidic chip that makes it possible to form a jet that splits into drops, the outer layer of which is the hydrogel solution and the core of which is the solution of step (a) comprising the induced pluripotent stem cells; these drops are collected in a calcium bath that crosslinks and/or gels the alginate solution to form the shell.

To improve the monodispersity of the cellular microcompartments, the hydrogel solution is preferentially charged with a direct current at (between 1 and 10 kV). A ring to ground may optionally be arranged at a distance from the tip of between 1 mm and 20 cm, preferentially 3 mm to 10 cm, even more preferentially 1 cm to 5 cm, from the tip in the plane perpendicular to the axis of the jet exiting the microfluidic injector (coextrusion chip), to generate the electric field.

According to the invention, it is necessary to generate capsules, the Internal part of which has an average radius or smaller radius of at least 100 μm. To generate capsules with such dimensions with a coextrusion chip (microfluidic injector or microfluidic chip), the invention in particular proposes modifying the flow rate of the coextruded solutions and the final opening of the co-extrusion chip. “Flow rate” means the flow rate of each solution arriving at the injector. “Final opening of the coextrusion chip” means the internal opening of the outlet channel of the chip.
for the flow rate: the method preferentially goes, for each coextruded solution for the standard capsule sizes known from the prior art, from a value of between 20 and 40 ml/h to a value of between 45 and 150 ml/h, preferentially between 45 and 110 ml per hour for a variant of the invention,
for the final opening diameter of the coextrusion chip (microfluidic injector), which goes from a value preferentially between 50 and 120 μm for the standard capsules sizes known from the prior art, to a diameter value of between 150 and 300 μm, preferentially between 180 and 240 μm.

Thus, according to a particular embodiment, the encapsulation of step (a) is carried out using a microfluidic injector whose final opening diameter is between 150 and 300 μm, preferably between 180 and 240 μm, and with the flow rate of each of the 3 solutions comprised between 45 and 150 mL/h, preferably between 45 and 110 mL/h.

According to a variant, step (a) of preparing a microcompartment can be carried out with stem cells, progenitor stem cells or cells capable of differentiating into liver cells.

Step (b) of cell differentiation is preferably carried out for at least 20 days, even more preferably at least 30 days.

During the differentiation of step (b), the cells organize themselves in an organization similar to a liver bud. This organization is typically marked by the structuring in the form of two cell sub-populations presenting two different organizations, one of the epithelial type (i.e. in the form of apicobasally polarized cell base and presenting tight junctions and the other of the mesenchymal type, that is to say, without apicobasal organization and presenting focal junctions.

The differentiation of the induced pluripotent stem cells of step (b) into liver microtissue can be carried out by any known differentiation method, as described by Raggi et al., Stem cell Report 2022 or Mallanna and Duncan, Curr Protoc Cell Bil, 2014.

According to one embodiment, steps (a) and/or (b) are implemented with permanent or sequential stirring. This stirring is important because it maintains the homogeneity of the culture environment and avoids the formation of any diffusion gradient.

Preferably, step (b) is carried out under hypoxia conditions; more preferably, the first 5 days of differentiations are carried out under hypoxia conditions.

Unexpectedly, differentiation under hypoxia conditions allows a better rate of expansion to be obtained.

Implementing the method according to the invention makes it possible to obtain microcompartments comprising at least 100, preferably at least 500, at least 800, at least 1,000, in particular at least 3,000 cells.

The method according to the invention is preferentially carried out in a closed chamber such as a closed bioreactor.

The invention also relates to a method for preparing a liver microtissue comprising the implementation of the steps of:
(a) preparing a three-dimensional closed cell microcompartment comprising, inside an outer hydrogel layer, induced pluripotent stem cells, and optionally extracellular matrix elements or a natural or synthetic extracellular matrix,
(b) inducing cell differentiation within the cell microcompartment, so as to obtain at least 3 different phenotypes of liver cells.
(c) removing the outer hydrogel layer to recover the liver microtissue of ovoid, cylindrical, spheroid or spherical or substantially ovoid, cylindrical, spheroid or spherical or ellipsoidal shape comprising at least 300 cells corresponding to at least 3 different liver cell phenotypes.

Step (c) consists in dissociating the microcompartment to obtain liver microtissue; the removal of the outer hydrogel layer can be carried out in particular by hydrolysis, dissolution, piercing and/or rupture by any means that is biocompatible, that is to say, non-toxic for the cells. For example, the elimination may be accomplished using phosphate-buffered saline, a divalent ion chelator, an enzyme such as alginate lyase if the hydrogel comprises alginate, and/or laser microdissection.

Step (c) of removing the outer hydrogel layer makes it possible to recover the liver microtissue of ovoid, cylindrical, spheroid or spherical or substantially ovoid, cylindrical, spheroid or spherical or ellipsoidal shape comprising at least 300 cells corresponding to at least 3 different liver cell phenotypes.

Preferably, the liver microtissue recovered in step (c) has an ellipsoidal shape.

The liver microtissue from step (c) may comprise at least 300, preferably at least 500, in particular between 300 and 14,000, even more preferably between 900 and 5,000 cells.

The liver microtissue from step (c) comprises at least one lumen, at least one cell in contact both with a lumen and with the medium outside the microtissue, and at least one cell surrounded only by cells.

Advantageously, the method according to the invention makes it possible to produce the liver microtissue according to the invention on a large scale so as to form a set of liver microtissues that can form up to 20% of the mass of the liver of the patient to be treated in less than 50 days, preferably in less than 40 days, in particular in less than 35 days.

In a preferred variant, the method according to the invention comprises at least one re-encapsulation of the liver microtissue after step (c), that is to say, at least two encapsulation cycles. Preferably, each encapsulation cycle corresponds to a pass. In this variant of the method (at least one re-encapsulation of the cells after step (c)), the number of cell divisions of the entire method (for all the passes) is at least 3, 4, 5, 6, 7, 8, 9, 10, 11, 12, 13, 14, 15, 20, 25, 30 cell division cycles.

In a method according to the invention, there may be several re-encapsulations, preferentially between 1 and 100, in particular between 1 and 10 re-encapsulation(s).

Each re-encapsulation can comprise:
a step that consists in dissociating the microcompartment or the series of microcompartments to obtain liver microtissue; the removal of the outer hydrogel layer can be carried out in particular by hydrolysis, dissolution, piercing and/or rupture by any means that is biocompatible, that is, non-toxic for the cells. For example, the elimination may be accomplished using phosphate-buffered saline, a divalent ion chelator, an enzyme such as alginate lyase if the hydrogel comprises alginate, and/or laser microdissection, and
a step of re-encapsulation of all or part of the liver microtissue in a hydrogel capsule. Re-encapsulation is a means suitable for increasing the cell amplification resulting from the pluripotent step, and reducing the risks of mutation.

Re-encapsulation consists in removing the external hydrogel layer, preferentially in resuspending, in a partially or totally dissociated manner, the cells which were in the form of cysts in the microcompartments, and in re-implementing the steps of the method.

According to one embodiment, the re-encapsulation comprises the following steps:
of hydrogel defining an inner part, the smallest radius or the average radius of said inner part being at least 300 μm;
(iv) culturing the resulting microcompartments in a culture medium
(vii) optionally recovering the resulting cellular microcompartments.

The compartmentalization in microcompartments makes it possible to eliminate the microcompartments containing even more mutated cells than the other capsules. Even if the mutated cells have a rapid growth they will reach the capsular confluence which will limit their multiplication. The compartmentalization also makes it possible not to contaminate the entire cell population, and also to eliminate the capsules containing mutant cells, at any time, in particular before a re-encapsulation step. This sorting may be done either by inline analysis, or by eliminating filled capsules more quickly than others, for example.

In one embodiment, at least one of the steps (preferably all the steps) is carried out at a temperature suited to the survival of the cells, included between 4 and 42° C. The temperature during cell proliferation must preferably be between 32 and 37° C. to avoid triggering mutations by lowering the performance of the repair enzymes. Likewise, preferably, the temperature must be low (ideally about 4° C.) to manage the stress on the cells in step (c).

At any time, the method according to the invention may comprise a step consisting of verifying the phenotype of the cells contained in the microcompartment. This verification can be carried out by identifying the expression by at least part of the cells contained in the microcompartment, of specific markers of the phenotype sought selected from:
the expression of alfa-fetoprotein, albumin and cytokeratin 19 to characterize hepatoblasts
the expression of alfa-fetoprotein and albumin and the absence of expression of cytokeratin 19 to characterize immature hepatocytes
the expression of albumin and the absence of expression of alfa-fetoprotein and cytokeratin 19 to characterize mature hepatocytes
the expression of cytokeratin and the absence of expression of albumin and alfa-fetoprotein to characterize cholangiocytes
the expression of CD31 to characterize endothelial cells
the expression of CD90 and CD73 to characterize mesenchymal stem cells.

The cellular microcompartments obtained according to the methods of the invention can then be frozen before any use. The freezing is preferentially carried out at a temperature of between −190° C. and −80° C. The thawing can be carried out by immersing the sealed freezing vessel (screwable ampoule or plastic pouch) in a warm water bath (37 degrees preferentially) so that the cells thaw quite rapidly. The microcompartments according to the invention before they are used may be kept at more than 4° C. for a limited time before they are used, preferentially between 4° C. and 38° C.

The invention especially promotes amplification with a high rate of expansion, which consequently reduces the culture time to obtain a very large number of functional lymphocytes.

### EXAMPLE

### Example 1: Differentiation Protocol

The liver microtissue according to the invention can be obtained from any known differentiation method.

To demonstrate this, the inventors have adapted 2 protocols known from the prior art. Protocol A described by Raggi et al., Stem cell Report 2022 and protocol B adapted from Mallanna and Duncan, Curr Protoc Cell Bil, 2014 and Raggi et al., Stem Cell Reports, 2022.

### Materials and Method

The IPSCs were cultured on T75 flasks coated with vitronectin and were passaged regularly using the dissociation reagent ReLeSR (Stem Cell Technologies). All experiments were performed with iPSCs between passes 20 and 26.

The encapsulation of the IPSCs was carried out in alginate microcapsules of similar size between the two protocols A and B and in the presence of extracellular matrix.

### For Protocol A:

Minimum diameter on the long axis=220 μm
Maximum diameter on the long axis=550 μm
Average diameter on the long axis=415 μm

### For Protocol B:

Minimum diameter on the short axis=200 μm
Maximum diameter on the short axis=385 μm
Average diameter on the short axis=295 μm

The IPSCs were first detached with accutase in small groups of 3 to 5 cells and resuspended at a concentration of 0.8 E6 cells/mL in a mix composed of 50% Matrigel and 50% mTESR1 medium containing 10 UM of Rhock inhibitor (Y-27632) and encapsulated by adapting the method and the microfluidic device described in Alessandri et al., 2016 (“A 3D printed microfluidic device for production of functionalized hydrogel microcapsules for culture and differentiation of human Neuronal Stem Cells (hNSC),” Lab on a Chip, 2016, vol. 16, no. 9, pp. 1593-1604). The encapsulated cells were resuspended in an mTESR1 medium containing 10 UM of Rhock inhibitor in a proportion of 0.2 mL of capsule for 1 mL of total medium. The cells were allowed to form cysts in the capsules for 96 h under stirring conditions, using a 30 mL ABLE bioreactor (Reprocell), at 37° C. and 5% CO2 with a change of medium every 24 h in mTESR1 medium.

Differentiation was initiated 96 h after encapsulation by passing through RPMI medium, containing 1 mM Ca2+, 1% KnockOut replacement serum (KOSR), B27 without insulin, 100 ng/ml Activin A and 3 μM CHIR-99021 for 2 days.

During the following 3 days, the medium was replaced by RPMI medium, containing 1 mM Ca2+, 1% KOSR, B27 without insulin and 100 ng/ml Activin A.

Then, during the following 5 days, the medium was replaced by an RPMI medium, containing for protocol A: 1 mM Ca2+, 1% KOSR, B27 without insulin, supplemented with 20 ng/ml of BMP-4, 5 ng/ml of bFGF, 1 μM of A83-01 (TGF beta pathway inhibitor) and 4 μM of IWP-2 (Wnt channel inhibitor).

For protocol B: 1 mM Ca2+, 1% KOSR, B27 without insulin, supplemented with 20 ng/ml BMP-4 and 5 ng/ml bFGF. From day 11 to day 15, the medium was composed of RPMI medium, containing 1 mM Ca2+, 2% KOSR, B27 with insulin, supplemented with 20 ng/mL HGF, 3 μM CHIR-99021, 5 ng/ml bFGF and 20 ng/ml BMP-4.

On day 16, the medium was changed to HCM medium without EGF (Lonza), supplemented with 20 ng/ml HGF, 3 μM CHIR-99021, 5 ng/ml bFGF, 20 ng/ml BMP-4, 20 ng/ml Oncostatin M, 10 μM Dexamethasone and 1% KOSR.

From day 20 and for the following 5 days, the medium was composed of HCM medium plus EGF, supplemented with 20 ng/mL Oncostatin M, 10 μM Dexamethasone and 1% KOSR.

From the 25th day, the medium was composed of HCM medium plus EGF, 10 μM Dexamethasone and 1% KOSR.

### Gene Expression Analysis

Gene expression analysis was performed by RT-Q-PCR.

qPCR was performed on a Roche LightCycler® 480 instrument. The expression of each gene was normalized by the expression of the reference genes YWHAZ, NONO and VCP. Data are expressed as 2\hat{ }delta Ct, where delta Ct=Ct of gene−average Ct of the reference genes.

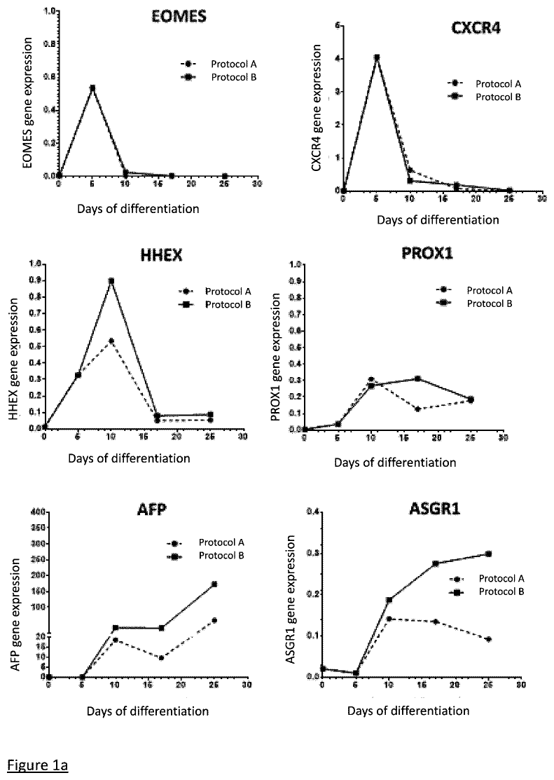

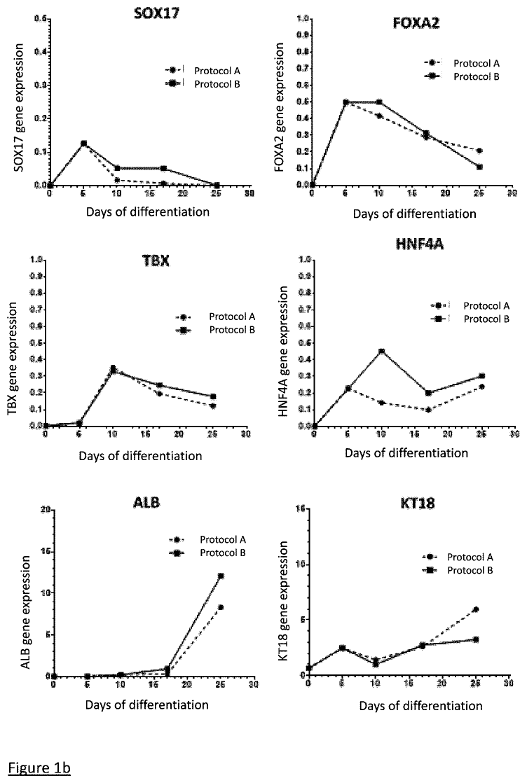

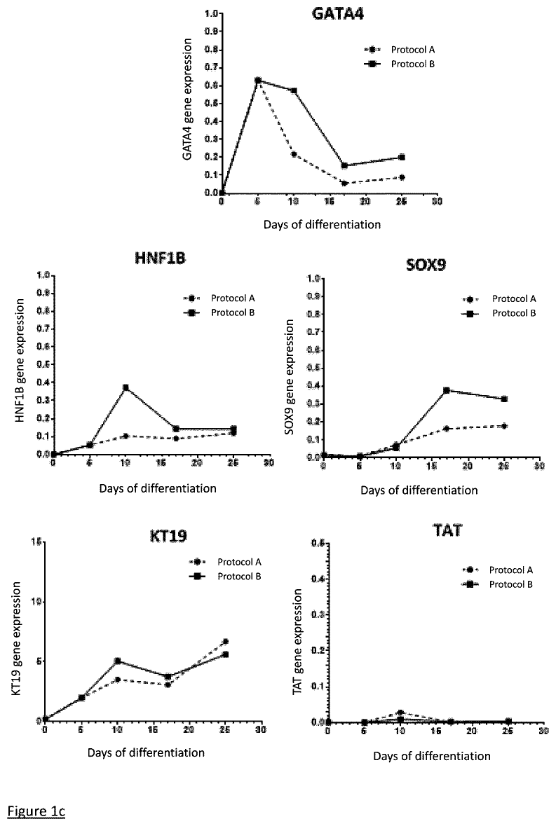

The results are shown in FIGS. 1a, 1b and 1c. These results show that the liver microtissue according to the invention can be obtained with any suitable differentiation method.

### Example 2: Differentiation into Hepatocytes within the Microcompartment According to the Invention Vs. In Two Dimensions

The IPSCs were encapsulated in alginate microcapsules with the following characteristics:
Minimum diameter on the long axis=450 μm
Maximum diameter on the long axis=685 μm
Average diameter on the long axis=575 μm
Minimum diameter on the short axis=313 μm
Maximum diameter on the short axis=546 μm
Average diameter on the short axis=423 μm

For the encapsulation of the IPSCs in alginate microcapsules of approximately 575 μm in average diameter and in the absence of matrix, the IPSCs were first detached with accutase in small groups of 3 to 5 cells and resuspended at a concentration of 10 E6 cells/mL in an mTESR1 medium containing 10 UM of Rhock inhibitor (Y-27632) and encapsulated by adapting the method and the microfluidic device described in Alessandri et al., 2016 (“A 3D printed microfluidic device for production of functionalized hydrogel microcapsules for culture and differentiation of human Neuronal Stem Cells (hNSC),” Lab on a Chip, 2016, vol. 16, no. 9, pp. 1593-1604). The encapsulated cells were resuspended in an mTESR1 medium containing 10 μM of Rhock inhibitor at 0.5 E6 cells/mL, the proportion of capsules relative to the medium not exceeding 20%. The cells were allowed to agglutinate in the capsules for 24 h under stirring conditions, using a 30 mL ABLE bioreactor (Reprocell), at 37° C. and 5% CO2.

Differentiation was initiated 24 h after encapsulation by passing through RPMI medium, containing 1 mM Ca2+, 1% KnockOut replacement serum (KOSR), B27 without insulin, 100 ng/ml Activin A and 3 μM CHIR-99021 for 2 days.

During the following 3 days, the medium was replaced by RPMI medium, containing 1 mM Ca2+, 1% KOSR, B27 without insulin and 100 ng/ml Activin A.

Then, for the next 5 days, the medium was replaced with RPMI medium, containing 1 mM Ca2+, 1% KOSR, B27 without insulin, supplemented with 20 ng/ml BMP-4 and 5 ng/mL bFGF. From day 11 to day 15, the medium was composed of RPMI medium, containing 1 mM Ca2+, 2% KOSR, B27 with insulin, supplemented with 20 ng/ml HGF, 3 μM CHIR-99021, 5 ng/mL bFGF and 20 ng/ml BMP-4.

On day 16, the medium was changed to HCM medium without EGF (Lonza), supplemented with 20 ng/ml HGF, 3 μM CHIR-99021, 5 ng/ml bFGF, 20 ng/ml BMP-4, 20 ng/ml Oncostatin M, 10 μM Dexamethasone and 1% KOSR.

From day 20 and for the following 5 days, the medium was composed of HCM medium plus EGF, supplemented with 20 ng/ml Oncostatin M, 10 μM Dexamethasone and 1% KOSR.

From the 25th day, the medium was composed of HCM medium plus EGF, 10 μM Dexamethasone and 1% KOSR.

The medium was changed daily from day 1 to day 25, and every other day from day 25.

### 2D Hepatocyte Differentiation

The iPSCs were dissociated into individual cells using accutase and placed on 6-well plates coated with Matrigel in mTESR1 medium containing 10 μM of Rhock inhibitor at a density of 1×105 cells/cm2. Differentiation was initiated 24 h after plating using the same differentiation protocol and medium composition as for 3D capsules.

### Gene Expression Analysis

Gene expression analysis was performed by RT-Q-PCR.

qPCR was performed on a Roche LightCycler® 480 instrument. The expression of each gene was normalized by the expression of the reference genes YWHAZ, NONO and VCP. Data are expressed as 2\hat{ }delta Ct, where delta Ct=Ct of gene-average Ct of the reference genes.

The comparison of gene expression between hepatocytes obtained in two dimensions versus three dimensions is shown in FIG. 2 in order to verify the induction of the endoderm during differentiation. The expression of the EOMES, CXCR4, FOXA2 and SOX17 genes shows that the differentiation indeed goes through a transitory stage of endodermal differentiation toward the definitive endoderm, which passage is expected to generate liver cells from pluripotent cells.

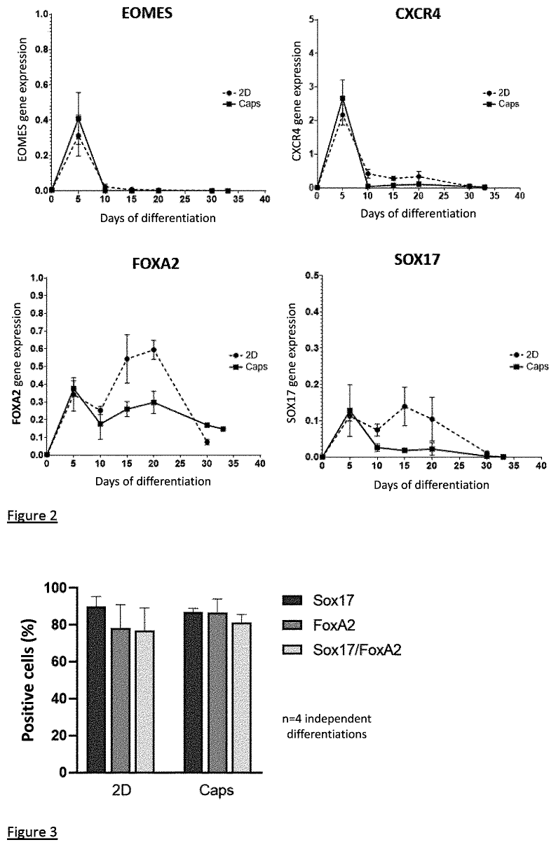

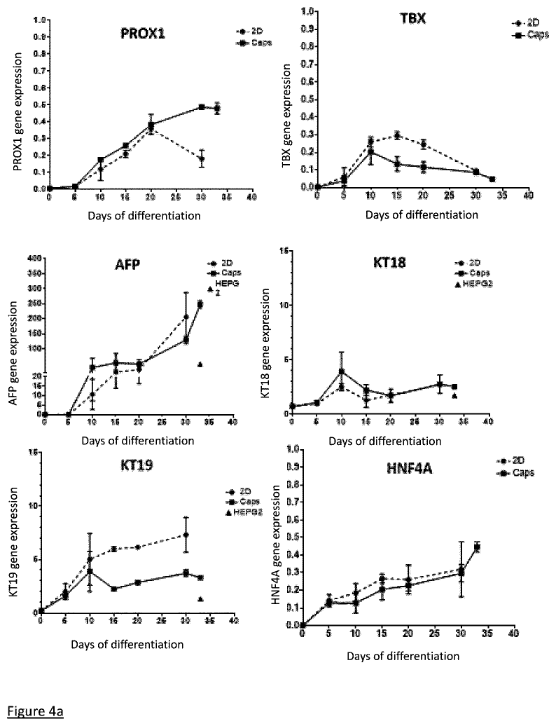

The analysis of the protein expression of endoderm markers was performed at day 5 and is shown in FIG. 3. These results show that the differentiation progresses according to a sequence in accordance with the expectations of those skilled in the art, that is to say, via the definitive endoderm and toward the liver bud.

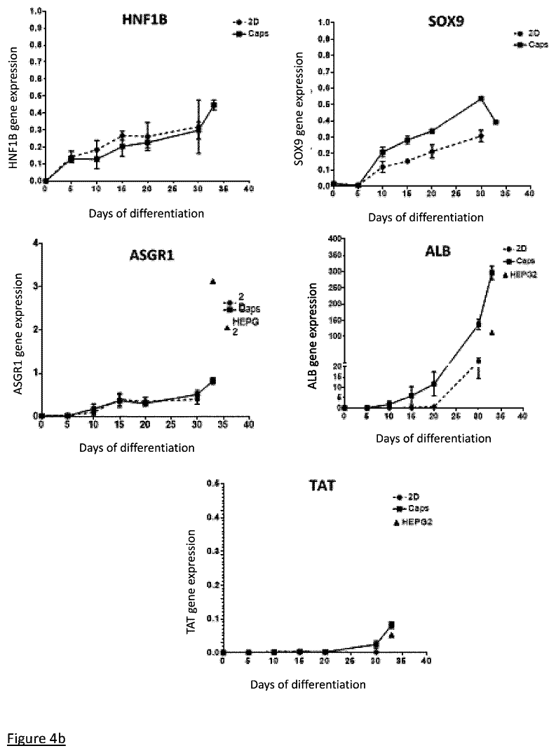

An analysis of gene expression during differentiation protocols is shown in FIGS. 4a and 4b. These results show that the differentiation progresses toward a composition that is consistent with the target of the cellular composition of the liver, in particular hepatocytes.

### Measurement of the Rate of Expansion;

The rate of expansion is measured by taking the ratio of the number of cells counted on day X of the culture divided by the number of cells at the start of the culture (day of encapsulation or day of placement in culture). The rate of expansion or amplification factor was measured during differentiation. The tracking of the rate of expansion during the differentiation process is shown in FIG. 5. A rate of expansion of at least 15 times, 20 days after the start of differentiation is found.

### Assessment of Albumin and Urea Production

To assess albumin and urea production, the conditioned medium was sampled periodically 24 hours (+2 hours) after the medium was changed and stored at −80° C. The levels of albumin and urea secreted into the medium were measured using the ELISA kit for human albumin (Invitrogen) and the Quantichrom urea assay kit (Gentaur), respectively, according to the manufacturer's instructions. The quantity of molecules secreted in 24 h was then normalized by the number of cells according to the count carried out after dissociation for each time point.

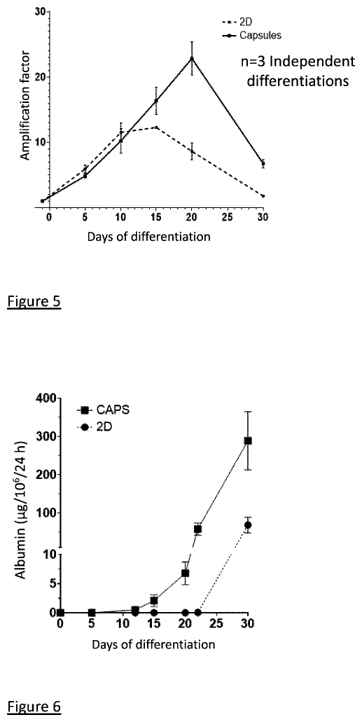

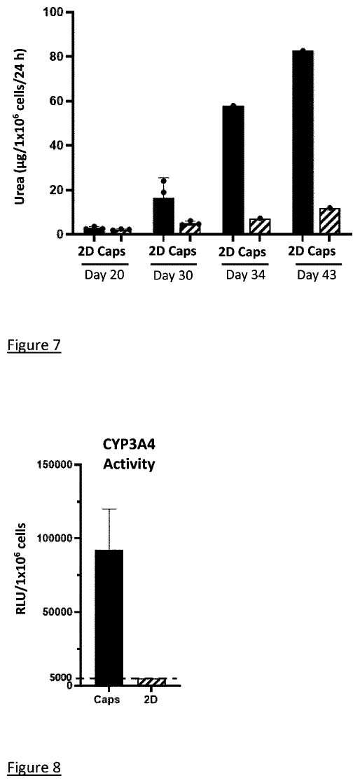

The results are shown in FIGS. 6 and 7. It is found that albumin secretion and urea production are greater in the microcompartments according to the invention.

### Measurement of CYP3A4 Activity:

Cyp3A4 activity was performed using the P450-Glo™ CYP3A4 assay with Luciferin-IPA (Promega) according to the manufacturer's instructions. Briefly, a sample of the 3D capsules containing the microtissues was decapsulated and the number of cells in a given volume of capsules containing the microtissues was determined. The decapsulated microtissues corresponding to the 1E5 cells, or to the 1E5 cells that were differentiated in 2D, were mixed with 50 μl of the substrate proluciferin P450-Glo 3 μM in a 96-well plate with a round bottom and incubated for 3 h at 37° C., 5% CO2. For each condition, 5 repetitions were performed, cell culture medium alone, and iPSCs were used as negative controls. Subsequently, 25 μl of the culture medium from each well was transferred to an opaque white 96-well luminometer plate and mixed with 25 μl of luciferin detection reagent. The plate was incubated for 20 minutes at room temperature and the luminescence was read using the Spectramax i3x microplate reader (Molecular devices) with an integration time of 1 second per well. The net signal was calculated by subtracting background luminescence values from control wells without cells.

The results are shown in FIG. 8. A significantly greater activity of CYP3A4 of the cells encapsulated in the microcompartment according to the invention is observed.

### Example 3: Morphological Study of the Microcompartment According to the Invention

The objective of this example is to characterize the morphology of cells within the microcompartment during differentiation.

### Histology

Liver microtissue samples were fixed with AFA fixative for 24 h at room temperature, washed with PBS and pre-embedded in histogel. After dehydration in successive baths of ethanol, acetone and xylene, the samples were embedded in paraffin and sectioned with a microtome to a thickness of 5 μm.

### Hematoxylin-Eosin-Saffron (HES) Staining

After paraffin removal, the sections were stained with Harris hematoxylin and eosin G. After dehydration, the sections were stained with Saffron and mounted with Entellan. The cellular cytoplasm is stained in pink, the nuclei in violet-blue and the extracellular matrix in yellow-pink.

### PAS (Periodic-Acid-Schiff) Staining

After paraffin removal, sections were pretreated with 1% periodic acid, followed by sequential staining with Schiff's reagent and Mayer's hematoxylin. The glycogen is stained pink and the nuclei blue-violet.

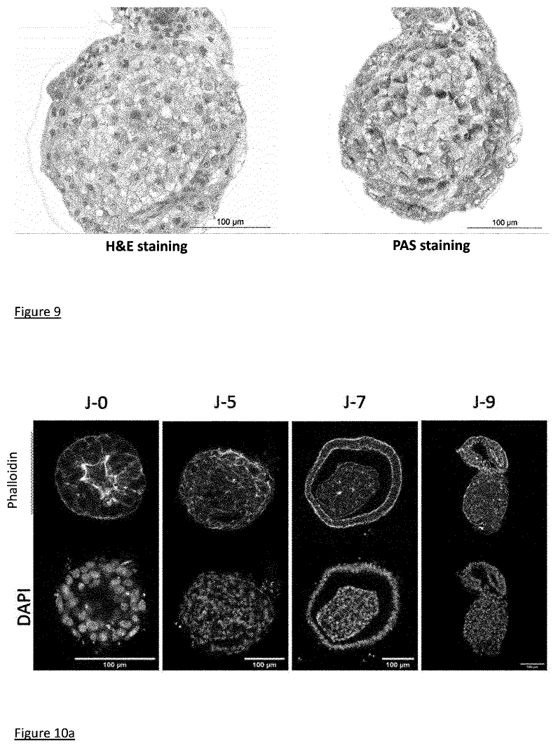

The images resulting from the staining are shown in FIG. 9. The characteristic cubic shape of hepatocytes can be clearly seen, as well as the presence of glycogen granules within the microcompartment.

### Immunofluorescence on 3D Microtissues

The microtissues were fixed inside the capsules in a solution of 4% paraformaldehyde in PBS with calcium for 1 hour at room temperature. After being rinsed with Ca2+-free PBS to remove the capsules, they were permeabilized in 1% Triton X-100 for 30-60 minutes at room temperature. The microtissues were incubated with the primary antibody solution for 72 h at 4° C. with stirring. After washing with PBS, they were incubated with a solution of marked secondary antibodies (Alexa Fluor, Life Technologies) and DAPI overnight at 4° C. with stirring and protected from light.

For structural imaging by phalloidin-DAPI, the staining was done while preserving the capsules (the decapsulation step before permeabilization was skipped, the washings were done with PBS containing calcium). The microtissues were incubated with DAPI and Phalloidin for 72 h at 4° C. with stirring.

After washing with PBS, the cells were mounted on a slide with a spacing of 0.5 mm. Images were acquired on a confocal microscope (SP5, Leica).

TABLE 1

| Antibody | Company | Ig Species | Dilution |
|---|---|---|---|
| AFP | DAKO | Rabbit | 1:2000 |
| ALB | Bethyl | Goat | 1:400 |
| CK19 | DAKO | Mouse | 1:100 |

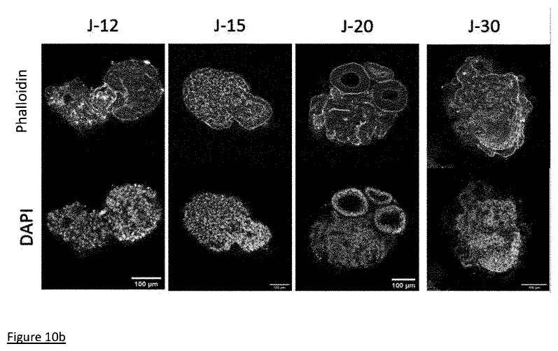

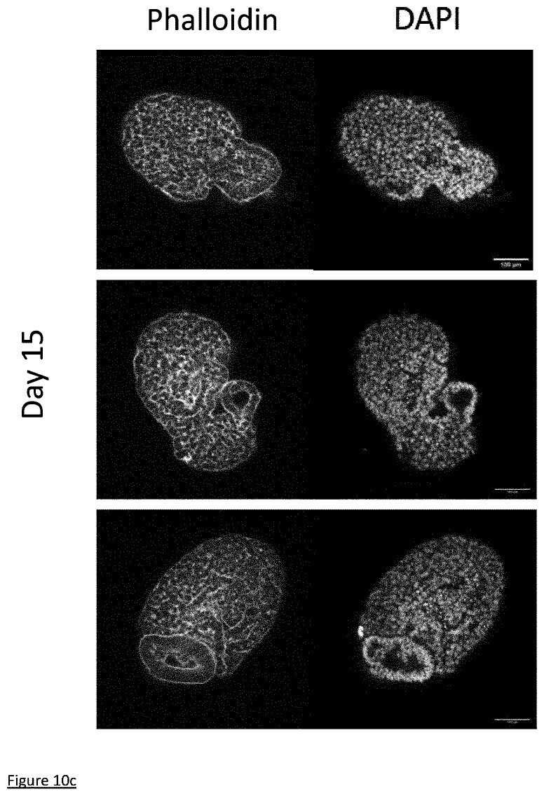

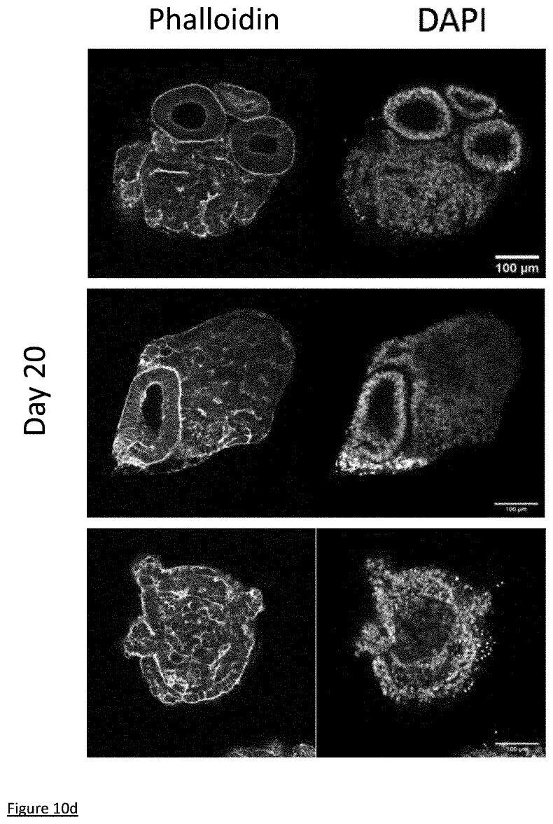

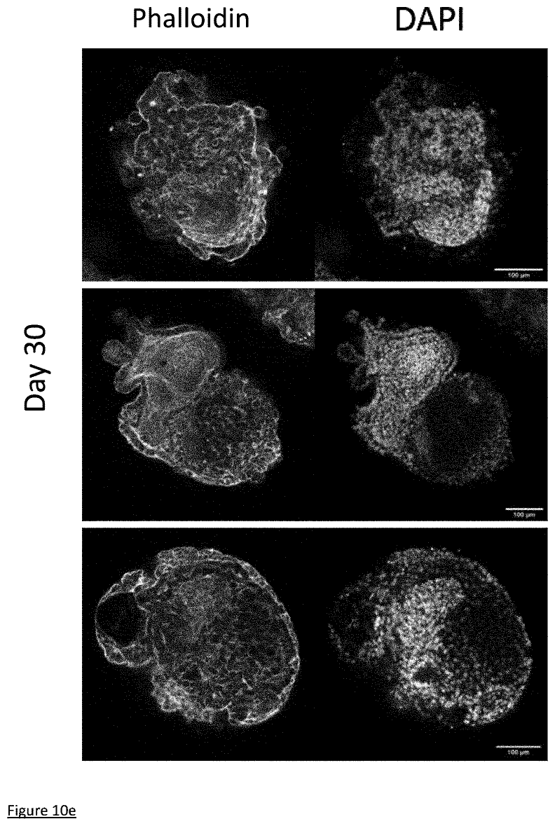

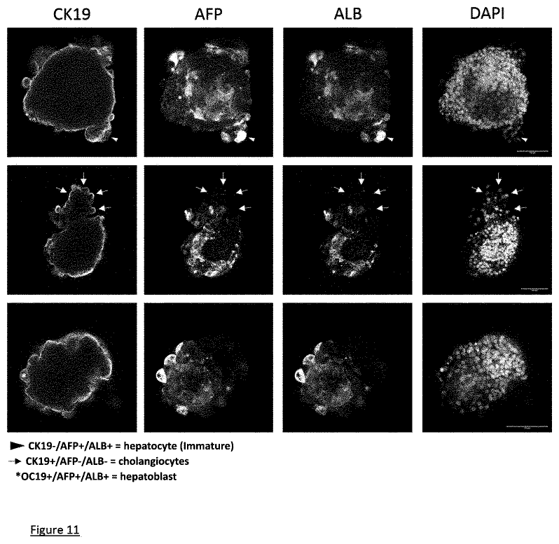

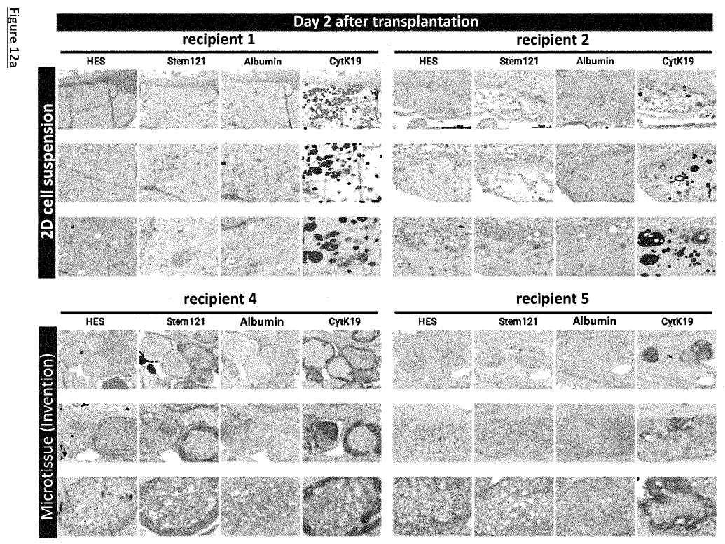

The evolution of the microcompartment's morphology was carried out for 30 days during the differentiation. These results are shown in FIGS. 10a to 10e. The presence of specific structures can be observed, such as the formation of a structure close to that of a liver bud on day 7 and a cellular organization characteristic of the liver microtissue according to the invention. Indeed, one can see at least one lumen, at least one cell in contact both with a lumen and with the medium outside the microtissue, and at least one cell surrounded only by cells. In addition to the study of the morphology, the inventors were able to characterize the presence of at least 3 different phenotypes of liver cells within the microcompartment, 30 days after the start of differentiation (FIG. 11).

Grafting of Microtissues According to the Invention and Assessment of Cell Survival after Transplantation in Mice Compared with Transplantation of Single-Cell Hepatocytes Obtained in 2D

The aim of this study is to inoculate immunocompetent mice with stem cell-derived hepatocytes, prepared as a cell suspension or microtissues according to the invention, with a view to assessing their survival and function in vivo.

The conditions of the study are described below:

### Animals:

The study was performed on 10-week-old female Balb/cByJ (Mus musculus) mice.

Seven (7) animals were housed in the animal house. Ventilation and air treatment were carried out by frequent renewals and the temperature was controlled at around 21-22° C. Humidity was maintained at around 50%. Artificial lighting was maintained for 12 hours per day. The quantity of and access to food (pellets) and drink (tap water) were monitored daily. The cages were changed once per week and the environment was enriched to minimize anxiety.

### Injection Method:

On day 42 of hepatic differentiation, the cells generated in 2D (according to example 2 “2D hepatocyte differentiation”) were dissociated into single cells and the hepatic microtissues generated according to the invention (according to example 1) were decapsulated.
Microtissue suspension: microtissues according to the invention obtained according to example 1, suspended in culture medium, were used: approximately 3×106 cells per tube diluted in 170 μL of basal medium. Prior to inoculation, 170 μL of Matrigel® was added to each tube.

In order to be able to perform subcutaneous inoculation of such a volume, that is, 340 μL, a skin incision was made in the right flank of the mouse to prepare a subcutaneous pocket. The microtissue suspension was then injected into this pocket using a pipette before the wound was closed with surgical glue to prevent diffusion of the suspension.
Cell suspension: cells obtained according to the protocol of example 2 “2D hepatocyte differentiation”, suspended in culture medium, were used: approximately 3×106 cells per tube in 120 μL of basal medium. Prior to inoculation, 120 μL of Matrigel® was added to each tube.

In order to be able to perform subcutaneous inoculation of this 240 μL volume, a skin incision was made in the right flank of the mouse to prepare a subcutaneous pocket. The cell suspension was then injected into this pocket using a pipette, before closing the wound with surgical glue to prevent diffusion of the suspension.
Control solution: 100 μL of basal medium solution (vehicle) was mixed with 100 μL of Matrigel®. This solution was injected subcutaneously using the same method as for the microtissues and cell suspension.

### Sampling:

Blood samples were collected by retro-orbital bleeding for serum preparation (approximately 80 μL) from all available mice:
Before cell inoculation, day 0, n=7
Two days after inoculation, day 2, n=7
Six days after inoculation, day 6, n=3

The grafts, including the surrounding skin in order to preserve the structure, were harvested as follows:
Two days after inoculation, day 2, n=4
   n=2 for cell suspension
   n=2 for microtissues
Six days after inoculation, day 6, n=3
   n=1 for cell suspension
   n=1 for microtissues
   n=1 for the vehicle

The grafts were harvested, fixed, paraffin-embedded, sectioned and stained with HES (hematoxylin eosin saffron) and with specific antibodies for the human cell marker Stem121 (detects human cytoplasmic proteins), the liver cell markers albumin and CytK19 (cytokeratin 19).

Prior to transplantation, the microtissues were also stained with HES, albumin and CytK19.

### Sampling:

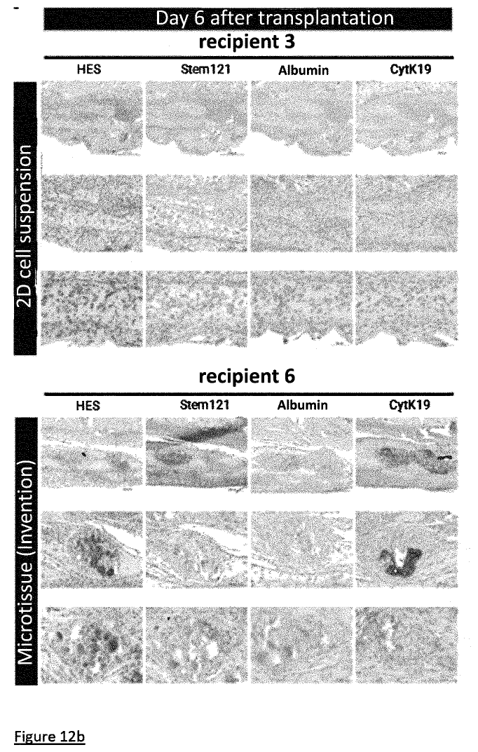

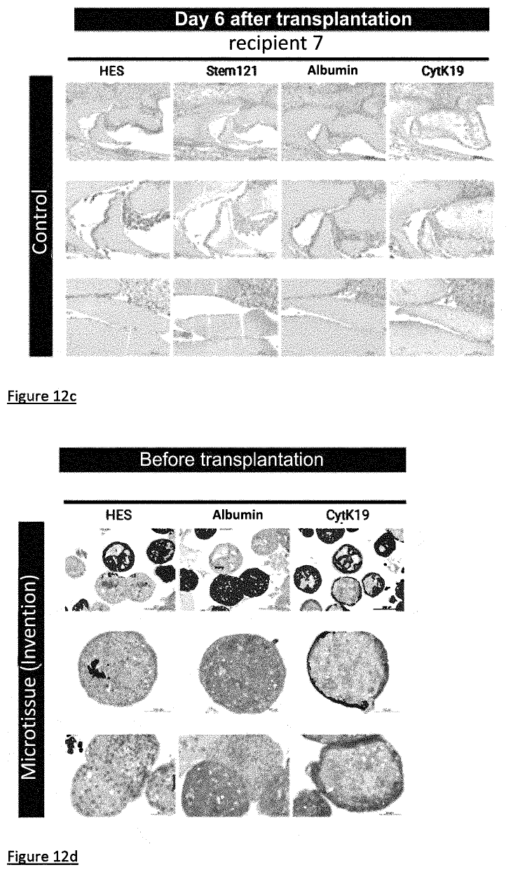

The images obtained by light microscopy (Leica DM2000 microscope) are shown in FIGS. 12a to 12d:
12a:
single-cell grafts on day 2 (recipients 1 and 2, top row) after transplantation into Balb/c mice,
microtissue grafts on day 2 (recipients 4 and 5, bottom row) after transplantation into Balb/c mice.
12b:
single-cell grafts on day 6 (recipient 3, top row) after transplantation into Balb/c mice,
microtissue grafts on day 6 (recipient 6, bottom row) after transplantation into Balb/c mice.
12c: tissue at the injection site with the vehicle alone (basal medium containing 50% matrigel) without a graft in Balb/c mice.
12d: microtissue according to the invention prior to transplantation.

Histological analysis of the grafts on day 2 after transplantation revealed the presence of round-shaped microtissues positive for the human cell marker Stem 121 in mice transplanted with the liver microtissues according to the invention, confirming that the microtissues detected are of human origin. Some of the cells making up the microtissues were positive for the hepatocyte marker albumin, and some cells, mainly those at the periphery of the microtissues, were positive for the cholangiocyte marker CytK19. Cell organization and the compartmentalization of cells expressing albumin and CytK19 after in vivo transplantation were similar to those observed in microtissues prior to transplantation.

In mice transplanted with 2D-generated single cells, only rare cells positive for the human cell marker Stem 121, albumin and cytokeratin 19 were detected on day 2 after transplantation.

The presence of cells expressing Stem121 and CytK19 was still detectable on day 6 in mice transplanted with the microtissues, no Stem 121- or CytK19-positive cells were present in mice transplanted with the single cells. Finally, in the area of immune cell infiltration in mice transplanted with the vehicle alone (control), no Stem121-, albumin- or cytokeratin-19-positive cells were detected.

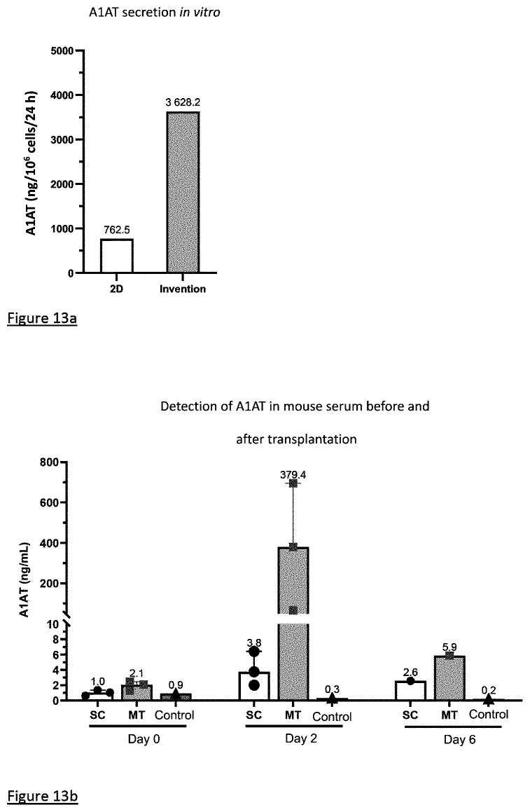

In addition, the presence of human A1AT was analyzed in the serums of transplanted Balb/c mice (analyses performed pre-transplantation (day 0), on day 2 and on day 6 after transplantation). The presence of human A1AT (alpha-1 antitrypsin) was analyzed by ELISA. A1AT secretion by cells and microtissues prior to transplantation was also analyzed. The results obtained are shown in FIGS. 13a and 13b:
13a: graphical representation of A1AT secretion prior to transplantation of 2D (2D) differentiated single cells and microtissues according to the Invention.
13b: graphical representation of A1T detection in Balb/c mouse serum pre-transplantation (day 0), on day 2 and on day 6 after transplantation with 2D differentiated single cells (SC), with microtissues according to the invention (MT) and with the vehicle alone (control).

These results show that liver microtissues according to the invention are able to secrete a higher level of A1AT than 2D differentiated cells. Consistent with the histological observation, the concentration of human A1AT is higher in the serum of mice transplanted with the microtissues according to the invention than in that of mice transplanted with single cells. Human A1AT concentration is lower, or even undetectable, on day 0. It is highest on post-transplant day 2 and decreases on day 6, but is still significant and higher than that of 2D differentiated single cells. These data are consistent with the histological results and show that the microtissues according to the invention persist over time and are more functional after transplantation than 2D differentiated single cells, even in an immunocompetent mouse model.

Overall, these results show that liver microtissues according to the invention and grafted as 3D objects survive after grafting in immunocompetent mice, and have a better and longer survival after grafting than 2D-generated liver cells grafted as a single cell suspension.

1. A three-dimensional liver microtissue, comprising at least 3 different phenotypes of liver cells, characterized in that all the cells of the microtissue have all been obtained from induced pluripotent stem cells encapsulated in a single three-dimensional closed microcompartment.

2. The liver microtissue according to claim 1, characterized in that the liver microtissue comprises immature hepatocytes, mature hepatocytes and cholangiocytes.

3. The liver microtissue according to claim 1, characterized in that the liver microtissue comprises at least:
immature hepatocytes characterized by the expression of alfa-fetoprotein and albumin and the absence of expression of cytokeratin 19
mature hepatocytes characterized by the expression of albumin and the absence of expression of alfa-fetoprotein and cytokeratin 19
cholangiocytes characterized by the expression of cytokeratin and the absence of expression of albumin and alfa-fetoprotein.

4. The microtissue according to claim 1, characterized in that the liver microtissue comprises cells expressing CD73 and CD90.

5. The liver microtissue according to the preceding claim 4, characterized in that the cells expressing CD73 and CD90 are mesenchymal stem cells.

6. The liver microtissue according to one of the preceding claims claim 1, characterized in that the liver microtissue comprises at least liver cells, of which between 20 and 60% of the liver cells are cells expressing cytokeratin 19.

7. The liver microtissue according to claim 1, characterized in that all the cells of the microtissue have all been obtained from the differentiation of at least one cyst of induced pluripotent stem cells encapsulated in a single three-dimensional closed microcompartment.

8. The liver microtissue according to claim 1, comprising:
at least one lumen,
at least one cell in contact with both a lumen and with the medium outside the microtissue, and
at least one cell surrounded only by cells.

9. The liver microtissue according to claim 1, characterized in that it has an ellipsoidal shape.

10. The liver microtissue according to claim 1, characterized in that the liver cells are polarized.

11. The liver microtissue according to claim 1, characterized in that it comprises between 50% and 99% liver cells, of which between 20% and 60% are cholangiocytes, and between 1% and 20% are cells expressing CD73 and CD90.

12. The liver microtissue according to claim 1, characterized in that it has a diameter or a smallest dimension of between 100 μm and 300 μm.

13. The liver microtissue according to claim 1, characterized in that it has a largest dimension of between 500 μm and 700 μm.

14. The liver microtissue according to claim 1, characterized in that it comprises between 300 and 14,000 cells.

15. The liver microtissue according to claim 1, characterized in that it comprises at least one bile duct.

16. The liver microtissue according to claim 1, characterized in that it comprises at least one glycogen granule.

17. The liver microtissue according to claim 1, characterized in that the liver cells are chosen from mature hepatocytes, immature hepatocytes, hepatoblasts, cholangiocytes and mixtures thereof.

18. The liver microtissue according to claim 1, characterized in that it expresses CYP3A4 monooxygenase with an activity of at least 75,000 RLU per million cells and/or produces at least 18 μg urea per million cells per 24 hours.

19. A three-dimensional closed microcompartment comprising an outer hydrogel layer defining an inner part, said inner part comprising at least one liver microtissue according to claim 1.

20. The cell microcompartment according to claim 19, characterized in that the microcompartment is obtained 20 days after the encapsulation of 1 to 200 induced pluripotent stem cells in the inner part defined by the outer hydrogel layer.

21. The cell microcompartment according to claim 19, characterized in that it exhibits a rate of expansion of at least 15 times, 20 days after the start of differentiation.

22. The cell microcompartment according to claim 19, characterized in that the thickness of the outer layer is variable and between 20 and 60 μm.

23. The cell microcompartment according to claim 19, characterized in that the outer layer comprises alginate.

24. The cell microcompartment according to claim 19, characterized in that it comprises extracellular matrix elements or a natural or synthetic extracellular matrix in the inner part between the outer layer and the liver microtissue.

25. The cell microcompartment according to claim 19, characterized in that it has a diameter or a smallest dimension of between 300 μm and 400 μm.

26. The cell microcompartment according to claim 19, characterized in that it has a largest dimension of between 400 μm and 600 μm.

27. An assembly of microcompartments comprising at least two three-dimensional cellular microcompartments, characterized in that at least one microcompartment is a microcompartment according to claim 19.

28. The assembly of microcompartments according to claim 27, characterized in that the microcompartments are arranged in a culture medium in a bioreactor.

29. A microtissue according to claim 1 or microcompartment comprising the microtissue for use thereof as a drug.

30. The microtissue according to claim 1 or microcompartment comprising the microtissue, for use thereof in the prevention or treatment of symptoms associated with liver failure.

31. The microtissue or microcompartment for use according to claim 30, wherein the liver failure is acute, chronic or acute-on-chronic liver failure.

32. The microtissue according to claim 1 or microcompartment comprising the microtissue, for use thereof in the treatment or prevention of metabolic diseases of the liver.

33. The microtissue according to claim 1 or microcompartment comprising the microtissue, for use thereof in the treatment or prevention of hepatic fibrosis and cirrhosis, steatosis, non-alcoholic steatosis, hepatitis, diseases linked to secretion of factor VIII and factor IX and VWF, Wilson's disease and hereditary hemochromatosis.

34. A method for preparing a microcompartment according to claim 19, comprising the following steps:
a) producing a three-dimensional closed cellular microcompartment comprising, inside an outer hydrogel layer, induced pluripotent stem cells, and optionally extracellular matrix elements or a natural or synthetic extracellular matrix,
b) inducing cell differentiation within the cell microcompartment, so as to obtain at least 3 different phenotypes of liver cells.

35. The method according to claim 34, characterized in that the cell differentiation method of step b) lasts at least 20 days.

36. The method according to claim 34, characterized that in step a) between 40 and 150 induced pluripotent stem cells are present in the microcompartment.

37. A method for preparing a liver microtissue according to claim 1, characterized in that it comprises:
implementing a method for preparing a three-dimensional closed microcompartment comprising an outer hydrogel layer defining an inner part, said inner part comprising at least one liver microtissue, and
removing the outer layer of hydrogel to recover the liver microtissue.
